(19) United States
(12) Patent Application Publication
(10) Pub. No.: US 2025/0213697 A1
(43) Pub. Date: Jul. 3, 2025

(54) INJECTABLE AND/OR SPRAYABLE, IN SITU POLYMERIZABLE COLLAGEN COMPOSITIONS FOR SUSTAINED DELIVERY OF THERAPEUTICS

(71) Applicant: SHANGHAI QISHENG BIOLOGICAL PREPARATION CO., LTD., Shanghai (CN)

(72) Inventors: Dale P. DeVore, Chelmsford, MA (US); Jiaxun Zhu, Shanghai (CN)

(21) Appl. No.: 18/398,968

(22) Filed: Dec. 28, 2023

(51) Int. Cl. A61K 47/42; A61K 9/08; A61K 31/167; A61K 31/4184; A61K 31/506; A61K 31/5575; A61K 38/12; A61K 38/18; A61K 47/18; A61K 47/26; A61K 47/64; A61K 49/00; A61K 51/08

(52) U.S. Cl. CPC A61K 47/42; A61K 9/08; A61K 31/167; A61K 31/4184; A61K 31/506; A61K 31/5575; A61K 38/12; A61K 38/1808; A61K 47/183; A61K 47/26; A61K 47/6435; A61K 49/0071; A61K 51/08

(57) ABSTRACT

Provided herein is injectable and/or sprayable, in situ polymerizable collagen-based compositions for sustained delivery of therapeutics (active agents, such as drugs, proteins, and other therapeutic agents). The compositions comprise (i) a neutral collagen solution, which is injectable or sprayable and capable of converting into a fibrous collagen mass or gel upon contacting with a physiological fluid or solution, or water; and (ii) a biologically or pharmaceutically active agent in the neutral collagen solution. The compositions are in the form of clear, transparent viscous solutions that instantaneously undergo gelation and polymerization when contacted by physiological fluids or solutions or water. The collagen solutions can be manipulated to provide various rates of sustained delivery of therapeutics.

### FIELD OF INVENTION

The present invention describes injectable and/or sprayable, in situ polymerizable collagen-based compositions for sustained delivery of active agents, such as drugs, proteins, and other therapeutic agents. The compositions are in the form of clear, transparent viscous solutions that instantaneously undergo gelation and polymerization when contacted by physiological fluids or solutions or water. The collagen compositions are chemically treated to produce temperature stable viscous solutions at neutral pH. Drugs, proteins, and other therapeutic agents are homogeneously mixed with the collagen solution in quantities to provide uniform sustained delivery for various therapeutic time intervals. Upon injection or spraying the solutions containing active agents rapidly undergo gelation and polymerization to form fibrous gel matrices providing a depot for sustained delivery of the active agents. The collagen solutions can be manipulated to provide various rates of sustained delivery and physiological resorption of the fibrous matrices.

### BACKGROUND

The development of in situ gelling drug delivery systems has received considerable attention over the past few years. In situ polymerizable compositions are drug delivery systems that are in solution form before administration and upon administration undergo gelation or polymerization to form a depot for sustained release of drugs or active agents. These delivery systems exhibit a number of advantages including reduced frequency of administration and improved patient compliance and comfort. In situ polymerizable gels may be administered by such as injectable, sprayable and intraperitoneal routes for a variety of application sites including, but not limited to, ocular, otic, nasal, skin, virginal tissues.

In situ polymerization or gel formation may be triggered by temperature modulation, pH change, presence of ions or ultra violet irradiation, from which the drug is released in a sustained and controlled manner. Various polymers that have been used for the formulation of in situ gels including gellan gum, alginic acid, carbomer xyloglucan, pectin, chitosan, poly(DL-lactic acid) (PDLLA), poly(DL-lactide-co-glycolide) and poly-caprolactone. The benefits of using biodegradable compositions are obvious.

Numerous properties of collagen favor its use as a biocompatible delivery agent (Simpson, Biomaterials in Reconstructive Surgery, In Collagen as a Biomaterial, C. V. Mosby Co., Ch. 11, pages 109-117, 1983; DeVore, Collagen as an Ophthalmic Biomaterial, In Encyclopedic Handbook of Biomaterials &amp; Bioengineering, Part B, Applications, Marcel Dekker, Inc., New York, Ch. 45, 1995; Silver and Garg, Collagen: Characterization, Processing and Medical Applications, In Handbook of Biodegradable Polymers, Eds. Domb, Kost, and Wiseman, Harwood Academic Publishing, Australia, Ch. 17).

Collagen compositions in various forms have been developed and used for drug delivery. These forms include solid inserts, eye shields, particles, films, sponges, gels and aqueous injections (Friess, W. Collagen-biomaterial for drug delivery. 1998. European Journal of Pharmacokinetics and Biopharmaceuticals. 45:113-136).

A number of issued and pending patents describe collagen compositions for drug delivery including U.S. Pat. No. 4,164,559 (Miyata et al) describing the application of succinylated collagen gels, U.S. Pat. No. 5,807,581 (Rosenblatt and Berg) describing injectable collagen compositions combined with crosslinking agents for delivering drug depots, and US 2011/0301131 (Fitzpatrick and Prior) describing a collagen fibrillar matrix implant for drug delivery. However, the performances of those materials for sustained delivery of the active agents are not fully satisfactory. For example, the collagen composition of Miyata et al. is not removable after drug release which, in some cases, are not desired. These collagen-based drug delivery systems (usually based on fibrillar collagen like sponges) increase the difficulty for implantation; and, the degradation-resistance approach for collagen-based material is by crosslinking which may sacrifice some biocompatibility. Hence, there is still need for novel collagen compositions with improved properties for sustained delivery of the active agents.

### SUMMARY OF THE INVENTION

In general, the present application relates to injectable and/or sprayable, collagen-based, in situ polymerizable systems for sustained delivery of active agents, such as drugs, proteins, or other therapeutic agents including cells. The present application also features a method for delivering active agents (such as chemical drugs, proteins, therapeutic agents, radio-active reagents) to a tissue site, the method comprises: (a) providing an active agent-collagen composition comprising the active agent in a neutral collagen solution (such as by mixing the active agent homogeneously with the neutral soluble collagen); and (b) administering the solution to the tissue site whereby, upon contact with the tissue, the solution is converted to a gel and the agent is released at a therapeutic level for the desired time period.

The collagen compositions can be designed to provide resorption ranges from a few hours to months. The rate of collagen resorption can be controlled by the form of collagen extracted or prepared, by the degree of collagen crosslinking, by adding insoluble collagen, and by adjusting the collagen concentration. Therefore, collagen compositions can be prepared in various forms or can be chemically modified to obtain formulations of the desired resorption time while still retaining collagen's hydrophilic and non-immunogenic properties.

The drug releasing property can both be adjusted by collagen composition and drug dosage forms. Additional drug dosage form design can be applied to different drugs and active agents, including but not limited to nano-crystallization, PEGlyzation, PLGA/PLA capsulation, lipid encapsulation, nanosphere or microsphere formation.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the polymerization of the injectable and/or sprayable rapidly polymerizable collagen composition (in situ polymerizable collagen):

FIG. 1A: in situ polymerizable collagen added to 0.15M NaCl; and

FIG. 1B: 1 minute after in situ polymerizable collagen addition to 0.15M NaCl.

FIG. 2 shows the sustained release of latanoprost from in situ polymerizable collagen gels.

FIG. 3 shows the release of Compstatin from in situ polymerizable collagen gels in 6 weeks (42 days).

FIGS. 4A and 4B show delivery of antibiotics from in situ polymerizable collagen:

FIG. 4A: the in situ polymerizable collagen/Voriconazole gel; and

FIG. 4B: the composition and polymerization of in situ polymerizable collagen/antibiotics gel in WFI. From left to right: Collagen/Cefuroxime composition; Collagen/Vigamox composition; and, Collagen/Voriconzaole composition.

FIG. 5 shows the formation of a transparent film after spraying the sprayable in situ polymerizable collagen for topically use of the present application to skin.

FIG. 6 shows a controlled release of lidocaine from in situ polymerizable collagen within two hours.

FIGS. 7A, 7B, and 7C show medical device coated with in situ polymerizable collagen/antibiotic composition and the antibacterial activity thereof:

FIG. 7A: in situ polymerizable collagen/Voriconazole coated hernia mesh parts;

FIG. 7B: in situ polymerizable collagen/Voriconazole coated bone screw; and

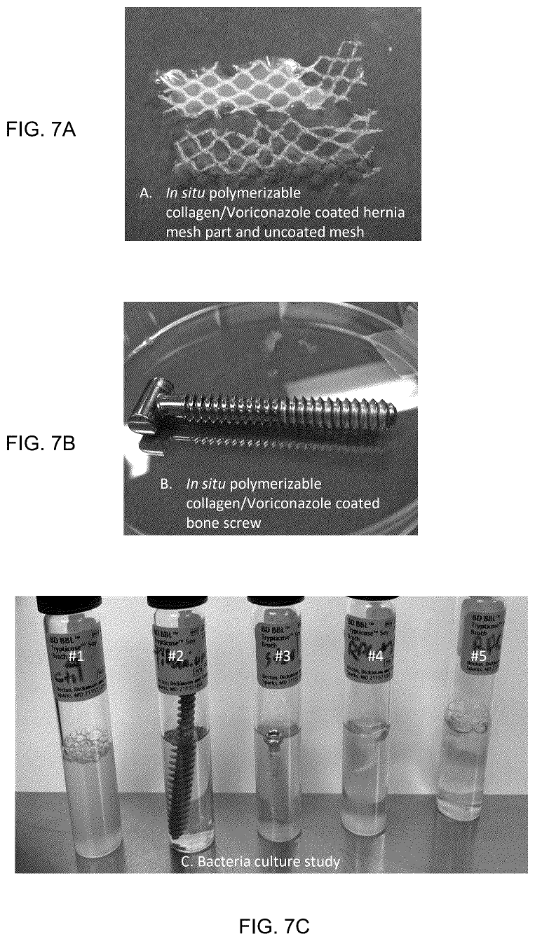

FIG. 7C: antibacterial study of in situ polymerizable collagen/Voriconazole coating:
#1: medium with bacteria;
#2: in situ polymerizable collagen/Voriconazole coated screw in medium with bacteria;
#3: in situ polymerizable collagen coated screw in medium with bacteria;
#4: in situ polymerizable collagen coated screw in medium without bacteria; and,
#5: in situ polymerizable collagen in medium without bacteria.

### DETAILED DESCRIPTION OF THE INVENTION

Described herein is a delivery system which uses an initially soluble collagen capable of rapid polymerization as a vehicle for delivery of active agents, such as drugs, proteins, and, therapeutic agents. The low viscosity of the initial soluble collagen allows homogeneous mixing with the drugs, proteins, therapeutic agents and administration by injecting or spraying to selected tissue sites.

The rapid polymerization characteristic of the collagen vehicle allows for targeted and sustained delivery of the active agent, at specific tissue sites. In addition, the dose and rate of delivery may be controlled by regulation of the concentration and in vivo release kinetics of agents.

Any collagen which exhibits the properties of an initially soluble state followed by rapid polymerization (preferably, within 180 seconds) may be used in the invention. This specific delivery system has been designed to polymerize when contacted by physiological fluids or solutions or water. Particularly preferred collagen compositions for use in the invention are described in such as DeVore & Eiferman (U.S. Pat. No. 5,492,135 assigned to Euclid Systems Corporation) and DeVore et al. U.S. Pat. No. 10,111,981B2. These collagen compositions are initially soluble in form and, upon exposure to physiological fluids or solutions or water in vivo or in vitro, undergo rapid polymerization. Such collagen solutions have been prepared at concentrations ranging from 1.0 mg/ml to over 70 mg/ml and at a pH ranging from 6.0-8.0. In some embodiments, the collagen composition is injected into a human or animal tissue or lumen, contacts with physiological fluid in vivo and undergoes rapid polymerization. In some embodiments, the collagen composition is combined with physiological solutions (such as PBS or saline) or water, then sprayed topically or through natural or artificial cavity canal and undergoes rapid polymerization.

In certain embodiments, the injectable or sprayable, polymerizable soluble collagen compositions are in the form of clear, viscous solutions at neutral pH. The compositions rapidly undergo polymerization involving initial gelation and subsequent fibrillization when contacted by or mixed with physiological fluids or solutions (preferably in neutral pH) or contact moist to form fibrous collagen matrices. Not intended to be bound by theory, it is believed that polymerizable soluble collagen composition comprises EDTA components associated with individual collagen molecules to protect the amine groups on collagen molecular units, and upon contacting with physiological fluids or solutions, the EDTA components associated with individual collagen molecules are diluted to allow protected amine groups on collagen molecular units to react as they would in normal tissue environment to form collagen fibrils. For simple dilution of the EDTA components, simple aqueous fluid might work and may suitable for forming a gel or coating in vitro or on a desired target surface.

In one embodiment of the present invention, a neutralized, acid solubilized collagen, which remains in solution at physiological temperatures, is used in the polymerizable collagen of the present invention. One method for initial treatment of the collagen to form such solution is disclosed in U.S. Pat. No. 5,492,135, which is incorporated herein by reference.

In some embodiments, the soluble collagen may be isolated and purified from animal sources including bovine and porcine tissues or may be a recombinant human collagen. The resulting acidic collagen solution is preferably extensively dialyzed against EDTA or EGTA or salt thereof, preferably disodium EDTA solutions to prevent premature collagen fibrillogenesis. During step-wise dialysis, the pH of the collagen solution is increased from acidic levels to a neutralized pH by adjusting the pH of the EDTA or EGTA or salt thereof (e.g., disodium EDTA) dialysis solutions using a basic solution, such as (sodium hydroxide, e.g., at a concentration of 0.5-5N, e.g., 1N). The resulting neutralized pH is preferably between about 6.8 and about 7.5.

Another method for forming the neural polymerizable or sprayable neutral collagen solution of the present application includes ultrafiltration (instead of dialysis) to remove addition salt or small molecule salt. A MWCO of 10,000-14,000 Da ultrafiltration membrane can be used in ultrafiltration. The ultrafiltration membrane can be ceramic membrane or polymer membrane package (including flat sheet membrane package, hollow fiber membrane module or spiral-wound membrane cartilage). 2.5 to 5 times volume of 0.01 M HCl solution was used to buffer the collagen solution and the volume of collagen solution remained the same before and after ultrafiltration.

At a neutralized pH, the collagen preparation does not undergo typical fibrillogenesis and remains clear and transparent until contacted by physiologic fluids or solutions or water. In certain embodiments, the composition comprises the acid soluble collagen in a concentration between 5 and 70 mg/ml (0.5-7.0% w/v), between 25 and 65 mg/ml (2.5-6.5% w/v) or between 20 and 40 mg/ml (2-5% w/v).

The EDTA or EGTA or salts thereof present in the composition can be is disodium salt, phosphate and/or citrate and/or carbonate and/or chloride salt, preferably disodium salt. The EDTA or EGTA or salts thereof remaining in the collagen composition after dialysis is preferably present in sufficient concentration to provide anti-collagenase activity sufficient to inhibit activity of tissue metalloproteinases and to provide bactericidal activity to assure sterility and potentially inhibit biofilm formation. In certain embodiments, the concentration of the EDTA or EGTA or salts thereof in the injectable composition is between 10 and 50 mM, between 25 mM and 40 mM, or between 30 mM and 35 mM.

In certain embodiments, the compositions of the present invention contain a polyol to bring the composition to physiologic osmolality and to aid in stabilizing the soluble collagen composition during storage. The polyol may also produce improved biocompatibility. Osmolality is preferably between 280 and 360 mmol/kg. In certain embodiments, the polyol is a sugar alcohol or other osmolality enhancer, and is preferably D-mannitol. In certain embodiments, the composition comprises the polyol in a concentration between 2.5% and 4%, or between 3.0% and 3.9%, or 3.5%.

Disaccharides, such as sucrose or fructose, may be included in the composition to aid in stabilization of the viscous collagen solution. In certain embodiments the disaccharides are added to a final concentration of between 50 mM to 500 mM, or 100 mM to 400 mM.

Upon exposure to physiologic liquids, tissues, or neutral pH solutions (such as water), the collagen solution polymerizes. Preferably, the composition undergoes rapid, nearly instant, gelation, followed by rapid fibrillogenesis to form a collagen fibril matrix. In certain embodiments, the composition initially polymerizes into a gel mass within 180 seconds after exposure to tissue, or within between 10 and 120 seconds or between 10 and 60 seconds after exposure to the tissue.

The collagen systems described herein may be used to deliver any biologically or pharmaceutically active agent, such as drug, protein, or therapeutic agent, including but not limited to, drugs to treat epidermal or mucosal injury, soft tissue damage, endometriosis, glaucoma, growth factors, steroids, antibiotics, inhibitors of angiogenesis, and an inhibitor of cell proliferation. In each case, the active agent is added to the collagen solution to a desired concentration, and the solution is administered to the appropriate tissue site. The active agent can be entrapped in the collagen, or covalently bound and crosslinked to the collagen. Combinations of two or more therapeutic agents may be administered simultaneously, or the active agent may be combined with other compounds which enhance the therapeutic agent's effect. In some embodiments, when the collagen formulation is used for spraying administration, a physically acceptable fluid such as physiological fluids, e.g., PBS or saline comprising the active agent is mixed with collagen solution. In some embodiments, the collagen formulation is used for spray-coating of a medical device, wherein the collagen solution comprising the active agent is mixed with a physically acceptable fluid, such as physiological fluids or solutions, e.g., PBS or saline or water and sprayed to a medical device to form a coating on the device. In some embodiments, for spray coating, the active/therapeutic component is mixed with the clear rapid polymerizing collagen composition and applied to the tissue to be treated. The rapidly polymerizing collagen rapidly undergoes fibril formation upon interaction with physiological fluids in the treated tissues. To facilitate polymerization, physiological buffer solution may be added during the delivery process.

If desired, the in vivo or in vitro stability of the collagen formulation following injection or spray can be prolonged by increasing its resistance to digestion and degradation. This can be achieved by increasing the crosslinking in the collagen molecules by either adding a fibrillar component to the collagen solution or by chemically inducing a limited crosslinking reaction. An active agent can also be chemically bound and crosslinked to the collagen, in soluble, partially soluble, or intact fibrillar form. Both collagen-collagen crosslinks and therapeutic agent-collagen crosslinks effectively slow the release of the therapeutic agent, and, thus, extend the duration of delivery. In some embodiments, an active agent is mixed with the collagen solution without crosslinking.

Any appropriate fibrillar component may be utilized to increase the in vivo stability of the collagen. For example, fibrillar collagen may be reconstituted collagen prepared from animal sources, such as bovine hide, using methods described in Borel and Randoux (Frontiers in Matrix Biology, Vol. 10, pages 1-58, In Methods of Connective Tissue Research, Eds. Robert, Moczar, and Moczar, S. Karger, Basel, 1985). Fibrillar collagen may also be prepared from human or animal tissue sources using methods described in Kelman and DeVore (U.S. Pat. Nos. 4,969,912 and 5,322,802). The concentrations of the fibrillar collagen component may range between 0.1-2.0% fibrillar collagen solids (w/v).

There are many potential chemical “stabilizing” compounds for use in chemically cross-linking the collagen molecules and the therapeutic agents, e. g., crosslinking collagen amine groups together or crosslinking collagen amine groups to amine, carboxyl, phenol, sulfonyl, or carbohydrate groups of therapeutic agents. Suitable crosslinking compounds include bifunctional acylation agents, including anhydrides, acid chlorides, sulfonyl chlorides, e. g., 1,2,3,4-cyclobutanetetracarboxylic dianhydride, tetrahydrofuran-2,3,4,5-tetracarboxylic dianhydride, 1,2,4,5-benzenetetracarboxylic dianhydride, ethylenediaminetetraacetic dianhydride, bicyclo (2,2,2) oct-7-ene-2,3,5,6-tetracarboxylic dianhydride, glutaryl dichloride, adipoyl chloride, 3-methyladipoyl chloride, pimeloyl chloride, terephthaloyl chloride, isophthaloyol dichloride, phthaloyl dichloride, 1,4-phenylene bis (chloroformate), 2,4-mesitylenedisulfonyl chloride, 2,6-naphthalenedisulfonyl chloride, malonyl dichloride, homobifunctional amine cross-reactive cross-linkers, such as homobifunctional imidoesters and homobifunctional N-hydroxysuccinimidyl, and heterobifunctional crosslinkers (Pierce, Rockford, IL).

The collagen-therapeutic agent delivery system described herein is ideally suited to provide sustained delivery of therapeutic agents for ocular, otic, and nasal treatments, such as sustained delivery of Latanoprost for treatment of glaucoma.

The collagen-therapeutic agent delivery system described herein is also ideally suited to provide sustained delivery of therapeutic agents for acute or chronic wound treatment, such as sustained delivery of EGF for treatment of burns.

In addition, the collagen-therapeutic agent delivery system described herein is ideally for local treatment through orifices, such as sustained delivery of letrozole to treat endometriosis.

In preferred embodiments, the composition is administered by injection, the tissue site is ocular (sclera, vitreal, conjunctival, sub tenon's, retinal), otic (cochlea, the endolymphatic sac/duct, the vestibular labyrinth, and all of the compartments and connecting tubes which include these components), nasal (sinus tissues), bone, cartilage, meniscus, tendon, ligament, or dermis.

In other preferred embodiments, the solution is administered by spaying, the tissue site is epidermis, dermis acute or chronic wounds, mucosa or submucosa wounds or disorder, and soft tissue wounds during surgery.

In other preferred embodiments, the composition is administered through orifices, the tissue site including respiratory tract, digestive tract, genital tract.

In other preferred embodiments, upon administration of the solution, the solution is converted to a gel or polymerized into a collagen fibrillar mass within 180 seconds, more preferably, within 120 seconds, most preferably, within 90 seconds. Preferably, the collagen-based solution is at a concentration of between 0.1-10%, more preferably, 0.3-7%, and most preferably between 0.5-5% collagen solids (w/v).

In addition, it may be desirable to administer a collagen-antibiotic drug depot or a combination collagen-antibiotic-steroid depot for sustained delivery following cataract surgery.

These examples are provided for the purpose of illustrating the invention and should not be construed as limiting.

The duration of delivery of the therapeutic agent can be extended by increasing collagen concentration to reduce diffusion of the drug, active agent, or protein out of the gel or fibrillar mass, by ionically binding the drug, active agent, or protein to the collagen molecules, or by short-time exposure of the collagen-based solution to ultraviolet irradiation to initiate limited crosslinking.

### Definitions

By “collagen” is meant all forms of collagen including those which are natural presented, isolated, have been processed or modified. The collagen may be of human or animal origin or may be produced using recombinant techniques. The present invention can use these and other typed of collagen including natural collagen and various collagen derivatives.

By “in situ polymerizable collagen system” is meant a soluble collagen preparation which, at neutralized pH, does not undergo typical fibrillogenesis and remains clear and transparent, and upon contacting a physiologic fluid or solutions or water (e.g., after injected in situ or before spraying topically or to a medical device) undergoes polymerization.

The term “target” is used in its broad sense and meant any desired subject or thing for the delivery and/or release of the active agent, which can be biological or non-biological. The target may comprise but not limited to human or animal or tissues or parts thereof, including wounds; non-living things, such as a medical device, a cosmetic device, a surface to be treated (e.g., to form an antimicrobial surface) and so on. By “tissue” is meant an aggregation of similarly specialized cells in an organism, preferably, mammalian, and, most preferably, human, where the cells are exposed to the organism's extracellular fluid, and are united in performance of a function within an organism.

By “active agent” is meant a substance that has biological or pharmaceutical activity when applied to a living human or animal body. The activity may comprise therapeutic activity, diagnostic activity, preventive activity and so on. By “drug” or “therapeutic agent” is meant a substance that is administered to improve the condition of a tissue following injury or disorder as a result of disease or degeneration. By “protein” is meant a protein or polypeptide that is administered to improve the condition of a tissue following injury or a result of a disease or degeneration.

By “physiological fluids or solutions” is meant a flow or liquid which is a body fluid or a physiologically acceptable liquid, usually comprising one or more ions, such as those selected from Na⁺, K⁺, Ca²⁺, Mg²⁺, Cl⁻, HCO₃⁻, HPO₄²⁻ and SO₄²⁻. Physiological fluids or solutions can be one or more selected from (but not limited to) liquids in a human or animal body, such as blood, plasma, interstitial fluid, lymph fluid, cerebrospinal fluid; artificial liquids, such as saline, Ringer's solution and derivates thereof (e.g., lactated Ringer's solution, acetated Ringer's solution, compound acetated Ringer's solution, sodium bicarbonate Ringer's solution), hydroxyethyl starch (HES, such as 6% HES), dextran (such as dextran-40, dextran-70), Tyrode's solution, PBS, Hank's Balanced Salt Solution (HBSS), Earle's Balanced Salt Solution (EBSS), and so on.

By “in situ polymerizable collagen gel” is meant a gel formed by contacting the in situ polymerizable collagen with physiological fluid or solutions or water. In some embodiments, the in situ polymerizable collagen gel comprises an active agent or is to be added with an active agent.

The present invention provides a number of advantages. For example, the collagen delivery systems described herein are biocompatible, biodegradable, and stable in solution at neutral pH. The ability to manipulate the collagen in solution form allows for the homogeneous dissolution or dispersion of the therapeutic agents and the injectable administration of these agents through a fine gauge needle (e. g., a 30-gauge needle). In addition to the ease of application, injectable delivery also allows access to the administration site while minimizing invasive injury to surrounding tissues. The density of the collagen solution is sufficient to fill a bone defect or other specific delivery site and remain in place until gelation and fibril formation occurs, as well as to maintain the active therapeutic agent in the injection site for a period of time sufficient to promote the effect.

The presently claimed collagen delivery system also has the advantage of allowing for the adjustment of active agent concentrations to achieve the desired dose. Furthermore, the crosslinking of molecules in the therapeutic agent-collagen formulation can be adjusted to extend the duration of therapeutic agent release. After polymerization, the injectable collagen delivery system also provides a scaffold-like structure to support new tissue growth.

In addition to the above, the collagen delivery system also has the advantage of providing local delivery. Thus, when the active agent has toxic side effects, e. g., chemotherapeutic agents, administration to a specific injection site minimizes exposure to surrounding tissues and dramatically reduces the undesirable, unnecessary side effects caused to the surrounding tissues or the body as a whole, while facilitating delivery of potent doses to the targeted cells. The local delivery system further has the advantage of minimizing the total amount of therapeutic agent required, and thus the cost of treatment.

### Exemplary Embodiments

The present application provides the following exemplary embodiments:

In some aspects, provided herein is a composition comprising:
(i) a neutral collagen solution, which is injectable or sprayable and capable of converting into a fibrous collagen mass or gel upon contacting with a physiological fluid or solutions or water); and
(ii) a biologically or pharmaceutically active agent in the neutral collagen solution.

In some embodiments, the composition is an injectable and/or sprayable, in situ polymerizable collagen composition. In some embodiments, the source of collagen in the neutral collagen solution is selected from allogenetic, mammal hides or marine species or axolotl hides derived matrix. In some embodiments, the collagen in the neutral collagen solution is selected from full collagen or atelocollagen, or recombinant collagen peptides from microorganism, plants, insect cells or animal cells, or collagen mimic peptides. In some embodiments, the collagen is soluble at neutral pH, does not undergo fibrillogenesis in the solution, and polymerizes upon exposure to a tissue site in vivo or to a physiological fluid or solutions or water in vitro or ex vivo. In some embodiments, the pH of the neutral collagen solution is from about 6.5 to about 7.5. In some embodiments, the neutral collagen solution comprises an acid soluble collagen, EDTA or EGTA or salts thereof, and optionally a polyol. In some embodiments, the pH of the physiological fluid or solutions is from about 6.5 to about 7.5.

In some embodiments, the collagen in the neutral collagen solution is in a concentration between 5 and 70 mg/ml. In some embodiments, the salt of EDTA is disodium EDTA or phosphate and/or citrate and/or carbonate and/or chloride salt of EDTA. In some embodiments, the salt of EGTA is disodium EGTA or phosphate and/or citrate and/or carbonate and/or chloride of EGTA. In some embodiments, EDTA or EGTA or salt thereof is in a concentration between 10 and 50 mM. In some embodiments, the polyol is a sugar alcohol. In some embodiments, the polyol is in a concentration between 2.5% and 4% (w/v).

In some embodiments, the sugar alcohol is selected from D-mannitol. In some embodiments, the solution further comprises a disaccharide, fructose, or combinations thereof.

In some embodiments, the active agent is selected from a drug, a biological molecule, a therapeutic agent, a diagnostic agent. In some embodiments, the active agent is selected from a chemical drug or active ingredient, a biological drug or active ingredient, a radioactive substance, a chemiluminescent or bioluminescent active substance. In some embodiments, the active agent is in the form of an element, a compound, a composition, a polymer, an oligomer, a conjugate or combinations thereof. In some embodiments, the active agent is selected from the group consisting of a digestive tract drug, metabolic drug, blood and hematopoietic organ drug, cardiovascular system drug, skin drug, genitourinary system drug, hormone drug, systemic anti-infection drug, anti-tumor drug, immunomodulator drug, musculoskeletal system drug, nervous system drug, anti-parasitic drug, respiratory system drug, sensory organ drug. In some embodiments, the active agent is a drug for treating disease selected from a peptide or protein, a nucleotide drug, a steroid, an antibiotic, an angiogenic agent, an inhibitor of angiogenesis, or an inhibitor of cell proliferation, an immune inhibitor, an antibiotics, an enzyme inhibitor, a signaling receptor agonist, a signaling receptor antagonist, a signaling receptor blocker, a chemotherapy drug, a radioactive drug, a hormone drug, a toxin drug, or a cell-based product. In some embodiments, the active agent is selected from latanoprost, peptide, steroids, cyclosporin voriconazole, ¹²⁵-I, liarozole and EGF, atropine or botulinum toxin.

In some embodiments, the concentration of the active agent in the neutral collagen solution is ranged from 1 μg/ml to 100 mg/ml. In some embodiments, the active agent is solved or dispersed in the collagen solution, entrapped in the collagen, or covalently bound and crosslinked to the collagen. In some embodiments, the active agent is homogeneously mixed with the neutral soluble collagen solution.

In some embodiments, the composition is in a form suitable for administering to a target or a part thereof by injecting, spraying, topical application, coating or dispersing. In some embodiments, upon administered to a tissue site of a subject, the solution converted to a fibrous mass or gel carrying, delivering and/or sustained releasing the active agent. In some embodiments, upon administration, the composition converts to a fibrous mass or gel covering the tissue, blocking the vessels or canals or packing the wound. In some embodiments, upon administration, the composition converts to a fibrous mass or gel within 180 second. In some embodiments, the active agent is sustained released in at least 1 day, at least 2 days, at least 1 week, at least 2 weeks, at least a month, at least 2 months, at least 3 months, at least 6 months. In some embodiments, the tissue is selected from one or more of ocular, otic, nasal, bone, cartilage, meniscus, tendon, ligament, dermis or vagina.

In some embodiments, the target is a living human or animal or a part thereof, a medical device, a cosmetic device. In some embodiments, the physiological fluids or solutions or water is in a tissue site in vivo or is added to the collagen solution in vitro. In some embodiments, the tissue is selected from the group consisting of sclera, vitreal, conjunctival, sub tenon's, retinal, cochlea, the endolymphatic sac or duct, the vestibular labyrinth, and all of the compartments and connecting tubes which include these components, sinus tissues. In some embodiments, the tissue site is epidermis, dermis acute or chronic wounds, mucosa or submucosa wounds or disorder, and soft tissue wounds during surgery. In some embodiments, the tissue is or comprises orifices, respiratory tract, digestive tract or genital tract. In some embodiments, the physiological fluid or solution is a body liquid or an artificial physiologically acceptable liquid. In some embodiments, the physiological fluid or solution is selected from blood, plasma, interstitial fluid, lymph fluid, cerebrospinal fluid, saline, Ringer's solution and derivates thereof, hydroxyethyl starch (HES), dextran, Tyrode's solution, PBS, Hank's Balanced Salt Solution (HBSS), Earle's Balanced Salt Solution (EBSS).

In some aspects, provided herein is a method for sustained delivery of a biologically or pharmaceutically active agent to a target (such as a subject) in need thereof, wherein the method comprises: (a) providing a composition comprising the active agent in a neutral collagen solution, wherein the neutral collagen solution is injectable or sprayable and capable of converting into a fibrous collagen mass or gel upon contacting with physiological fluid or solutions or water; and (b) administering the composition to the target, whereby, upon contact with a physiological fluid or solutions, or water, the collagen solution converts to a gel or fibrous mass and the active agent is sustained released to the target.

In some embodiments, the target is a living human or animal or a part thereof, a medical device, a cosmetic device or a part thereof. In some embodiments, the target is a tissue site for treatment of a disease or wound via or in a tissue site. In some embodiments, the composition is an injectable and/or sprayable, in situ polymerizable collagen composition. In some embodiments, the source of collagen in the neutral collagen solution is selected from allogenetic, mammal hides or marine species or axolotl hides derived matrix. In some embodiments, the collagen in the neutral collagen solution is selected from full collagen or atelocollagen, or recombinant collagen peptides from microorganism, plants, insect cells or animal cells, or collagen mimic peptides. In some embodiments, the collagen is soluble at neutral pH, does not undergo fibrillogenesis in the solution, and polymerizes upon exposure to a tissue site in vivo. In some embodiments, the pH of the neutral collagen solution is from about 6.5 to about 7.5. In some embodiments, the neutral collagen solution comprises an acid soluble collagen, EDTA or EGTA or salts thereof, and optionally a polyol.

In some embodiments, the collagen in the neutral collagen solution is in a concentration between 5 and 70 mg/ml. In some embodiments the salt of EDTA is disodium EDTA or phosphate and/or citrate and/or carbonate and/or chloride salts of EDTA. In some embodiments the salt of EGTA is disodium EGTA or phosphate and/or citrate and/or carbonate and/or chloride salts of EGTA. In some embodiments EDTA or EGTA or salt thereof is in a concentration between 10 and 50 mM. In some embodiments the polyol is a sugar alcohol. In some embodiments the polyol is in a concentration between 2.5% and 4% (w/v).

In some embodiments, the sugar alcohol is selected from D-mannitol. In some embodiments the solution further comprises a disaccharide, fructose, or combinations thereof.

In some embodiments, the active agent is selected from a drug, a biological molecule, a therapeutic agent, a diagnostic agent. In some embodiments, the active agent is selected from a chemical drug or active ingredient, a biological drug or active ingredient, a radioactive substance, a chemiluminescent or bioluminescent active substance. In some embodiments the active agent is in the form of an element, a compound, a composition, a polymer, an oligomer, a conjugate or combinations thereof. In some embodiments the active agent is in a further controlled release form selected from the group consisting of polymer modification, polymer encapsulation, nano-crystallization, microsphere formation, lipid encapsulation, lipid modification, lyophilization, emulsification. In some embodiments the active agent is selected from the group consisting of a digestive tract drug, metabolic drug, blood and hematopoietic organ drug, cardiovascular system drug, skin drug, genitourinary system drug, hormone drug, systemic anti-infection drug, anti-tumor drug, immunomodulator drug, musculoskeletal system drug, nervous system drug, anti-parasitic drug, respiratory system drug, sensory organ drug. In some embodiments the active agent is a drug for treating disease selected from a peptide or protein, a nucleotide drug, a steroid, an antibiotic, an angiogenic agent, an inhibitor of angiogenesis, or an inhibitor of cell proliferation, an immune inhibitor, an antibiotics, an enzyme inhibitor, a signaling receptor agonist, a signaling receptor antagonist, a signaling receptor blocker, a chemotherapy drug, a radioactive drug, a hormone drug, a toxin drug, or a cell-based product or vaccine. In some embodiments the active agent is selected from latanoprost, peptide, steroids, cyclosporin voriconazole, ¹²⁵-I, liarozole and EGF, atropine or botulinum toxin.

In some embodiments, the concentration of the active agent in the neutral collagen solution is ranged from 1 μg/ml to 100 mg/ml. In some embodiments the active agent is solved or dispersed in the neutral soluble collagen solution, entrapped in the collagen, or covalently bound and crosslinked to the collagen. In some embodiments the active agent is homogeneously mixed with the neutral soluble collagen solution. In some embodiments the composition is in a form suitable for administering to a target by injecting, spraying, topical application, coating or dispersing. In some embodiments upon administered to a tissue site of a subject, the solution converted to a fibrous mass or gel carrying, delivering and/or sustained releasing the active agent. In some embodiments upon administration, the composition converts to a fibrous mass or gel covering the tissue, blocking the vessels or canals or packing the wound. In some embodiments upon administration, the composition converts to a fibrous mass or gel within 180 second. In some embodiments the active agent is sustained released in at least 1 day, at least 2 days, at least 1 week, at least 2 weeks, at least a month, at least 2 months, at least 3 months, at least 6 months. In some embodiments, wherein the tissue is selected from one or more of ocular, otic, nasal, bone, cartilage, meniscus, tendon, ligament, dermis or vagina.

In some embodiments, the physiological fluid or solutions, or water is in a tissue site in vivo or is added to the collagen solution in vitro. In some embodiments, the tissue is selected from the group consisting of sclera, vitreal, conjunctival, sub tenon's, retinal, cochlea, the endolymphatic sac or duct, the vestibular labyrinth, and all of the compartments and connecting tubes which include these components, sinus tissues. In some embodiments, the tissue site is epidermis, dermis acute or chronic wounds, mucosa or submucosa wounds or disorder, and soft tissue wounds during surgery. In some embodiments the tissue is or comprises orifices, respiratory tract, digestive tract or genital tract. In some embodiments the physiological fluid or solution is a body liquid or an artificial physiologically acceptable liquid. In some embodiments, the physiological fluid or solution is selected from blood, plasma, interstitial fluid, lymph fluid, cerebrospinal fluid, saline, Ringer's solution and derivates thereof, hydroxyethyl starch (HES), dextran, Tyrode's solution, PBS, Hank's Balanced Salt Solution (HBSS), Earle's Balanced Salt Solution (EBSS).

While specific embodiments of the active substances (such as the siRNAs), products, compositions and methods herein have been discussed, many variations of the invention will become apparent to those skilled in the art upon review of this specification and the claims. The full scope of the invention should be determined by reference to the claims, along with their full scope of equivalents, and the specification, along with such variations.

### EXAMPLES

### Example 1. Preparation of In Situ Polymerizable Collagen Solution

The base in situ polymerizable collagen was prepared using methods described by DeVore, et. al. in U.S. Pat. No. 10,111,981. Pure soluble Type I atelo-collagen was purchased from Sewon, Sunmax or Advanced BioMatrix, Inc. Sodium chloride was added to the soluble, pepsin-digested collagen solution (3 mg/mL) to a concentration of 0.8M to precipitate collagen. The white, opaque precipitate was recovered by centrifugation for 30 minutes at 3,500 rpm and concentrated to approximately 50 mg/mL by placement on filter paper. The concentrated collagen precipitate was placed in dialysis tubing with a MW cut off of 100,000 and dialyzed against 0.1 N HCl for 16-18 hours. The resulting clear, viscous, re-dissolved collagen concentrate was then dialyzed against 0.035M EDTA (ethylenediaminetetraacetic acid, disodium salt dihydate, SigmaUltra ˜99%). Dialysis was continued for 5 days with daily adjustment of pH from the starting pH of 4.5 to a final pH of 7.5. The final clear and viscous collagen concentrate was collected and centrifuged to remove air bubbles. The final clear, viscous collagen exhibited a pH of 7.4 and did not undergo fibril formation at room temperature. Collagen fibrillogenesis was not triggered by pH or temperature.

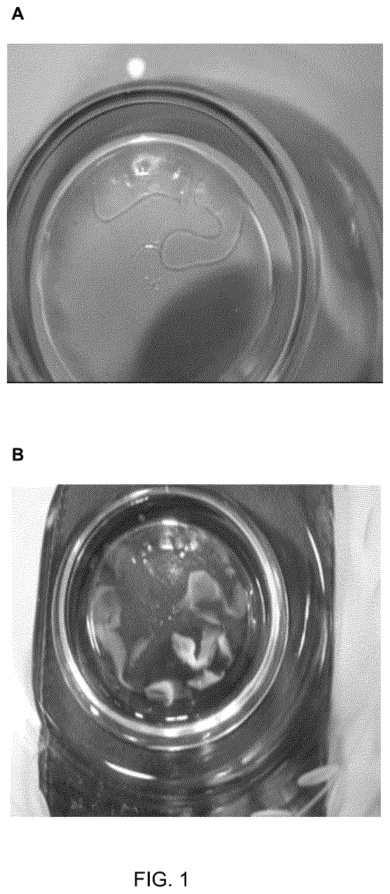

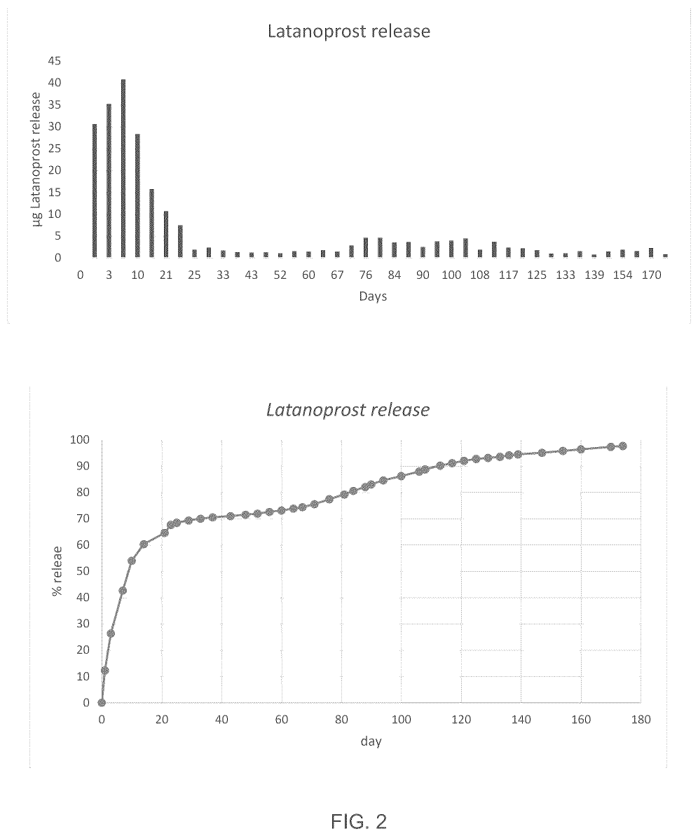

Evaluation of gelation and fibril formation: Aliquots of the in situ polymerizable collagen were injected into 0.8M sodium chloride at 37° C. and observed for the appearance of gel and fibrous collagen. The clear viscous collagen solution (FIG. 1A) formed a white, opaque collagen matrix (FIG. 1B) in less than 120 seconds.

### Example 2. Sustained, Long-Term Release of Latanoprost from In Situ Polymerizable Collagen

Latanoprost (13,14-dihydro-17-phenyl-18,19,20-trinor-prostaglandin F2a-1-isopropyl ester) was obtained from Sigma-Aldrich. Soluble, pepsin digested, bovine collagen for drug delivery formulations was purchased from Advanced Biomatrix. In situ polymerizable collagen was prepared according to Example 1 by extensive dialysis of salt precipitated collagen against 0.035M EDTA with step-wise increase in pH to 7.5. Latanoprost was dissolved in 95% ethanol and added to polymerizable collagen solution to provide gel depots containing 250 μg latanoprost/100 μL collagen. The solutions were incubated in 1.0 mL of physiological saline at 37° C. The entire 1.0 mL volume was removed at time periods ranging from 1 day to 174 days and replaced with 1.0 mL of fresh saline. Latanoprost concentration in saline aliquots was measured by HPLC analysis (Millennium Research Laboratories).

HPLC analysis showed a large initial release of Latanoprost from polymerizable gels added directly to saline solution followed by sustained release averaging 0.54 μg/day. Polymerizable gels partially pre-polymerized (which formed a weak collagen gel by drug mixing and incubation, but without releasing of EDTA) before placing in solution showed a reduced initial release followed and the total sustained release averaging 1.4 μg/day. In situ polymerizable collagen gels provided sustained in vitro release of Latanoprost for more than 30 days.

### Example 3. Release of a Complement Inhibitor Peptide from In Situ Polymerizable Collagen

Delivery of a complement peptide-Compstatin from in situ polymerizable collagen prepared according to Example 1 was studied in vitro. In vitro release was measured for 6 weeks to ascertain the optimum collagen formulation. Results showed bi-phasic release kinetics with an initial burst for the first 4 days followed by sustained release to 6 weeks (FIG. 3).

### Example 4. Delivery of Antibiotics from In Situ Polymerizable Collagen

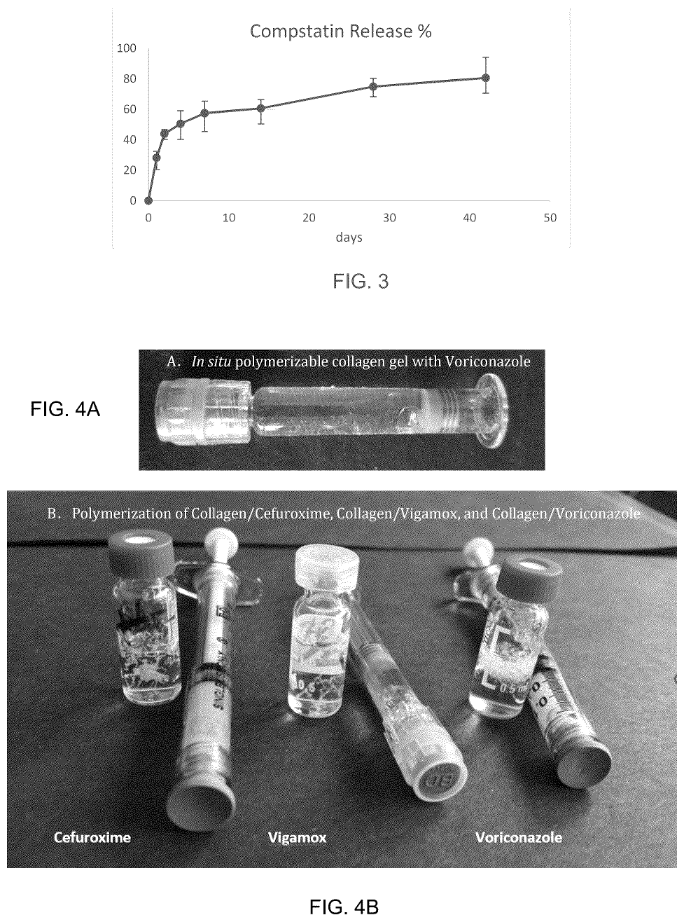

50 mg lyophilized and powdered (by mortar and pestle) Voriconazole was mixed with 200 μL of in situ polymerizable collagen solution prepared according to Example 1 by passing the powder into the collagen solution using two syringes attached with a female-female adaptor. The mixture appeared clear and transparent (FIG. 4A). Aliquots were injected into water for injection (WFI) using a 27-gauge needle. A control sample composed of in situ polymerizable collagen solution only was also injected into WFI. Similar fibril formation was observed in both the control sample and the antibiotics-containing samples (FIG. 4B). Both the control and Voriconazole supplemented samples exhibited collagen fibril formation although the time to fibril formation was slightly delayed for the Voriconazole sample.

Two other antibiotics, Cefuroxime and Vigamox, were mixed to in situ polymerizable collagen using the same method used for adding Voriconazole to the collagen to form a collagen/antibiotic composition, respectively. The in vitro polymerization tests show that antibiotic compositions do not blocking the fibril formation. The appearance of the mixture depends on the solubility of the antibiotics. Insoluble antibiotics or antibiotics/collagen mixing ratio exceeding solubility lead to the production of white powder dispersed in the gel (FIG. 4B), however, this change in appearance of collagen gel does not affect the polymerization of composition.

### Example 5. Delivery of Radio-Active Reagent for Local Radiotherapy

Specific radioactive isotopes (¹²⁵-I) were covalently complexed with in situ polymerizable collagen solution prepared according to Example 1 (Sample A), polymerized in situ polymerizable collagen obtained by adding in situ polymerizable collagen into 37° C. PBS over 2 hours (Sample B), or dispersed, collagen-rich tissue matrix (Zyderm I from INAMED Aesthetics) (Sample C) by fibrilization. Following labeling, all preparations were washed or dialyzed to remove unbound radiolabel.

Initial (radio)activity was measured after mixing, and control (radio)activity was measured after all preparations were washed or dialyzed to remove unbound radiolabel. All radioactivity was measured by two half-lives. The specific activity of bound radiolabel was 4.7 μCi ¹²⁵-I/mg Collagen in in situ polymerizable collagen solution (Sample A), 6.0 μCi ¹²⁵-I/mg Collagen polymerized in situ polymerizable collagen (Sample B), and 4.3 μCi ¹²⁵-I/mg Collagen in dispersed, collagen-rich tissue matrix (Sample C). It was shown to be highest in the in situ polymerizable collagen solution after polymerization. This preparation retained 86% of the initial activity and 93% of control activity. It is predicted that the quantity of ¹²⁵-I-collagen required to provide 0.5 mCi at time=0 would be 4 times less than that calculated at day 120. At time=0, approximately 25 mg of ¹²⁵-I-collagen would be needed. All preparations can be formulated to at least 25 mg/ml, and generally to as much as 50 mg/ml. At 50 mg/ml, only 500 μL of collagen preparation would be required to delivery 0.5 mCi of radioactivity. It is calculated that a collagen dose of 30-35 mg/ml would be required to deliver 0.5 mCi of ¹²⁵-I. This dose is suitable for use in brachytherapy procedures.

TABLE 1
¹²⁵-I activity per mg collagen at Day 120** (based on total
amount of radioactivity remaining in samples at D 120)

| | μCi ¹²⁵-I/ | μCi ¹²⁵-I/ | mg collagen needed for | |
|---|---|---|---|---|
| Sample | sample | mg Collagen | total 0.5mCi dose | |
| A | 11.7 | 4.7 | 106.4 | (soluble) |
| B | 15.0 | 6.0 | 83.3 | (reconstituted) |
| C | 10.7 | 4.3 | 116.3 | (dispersed) |

### Example 6. Sprayable In Situ Polymerizable Collagen for Topically Use

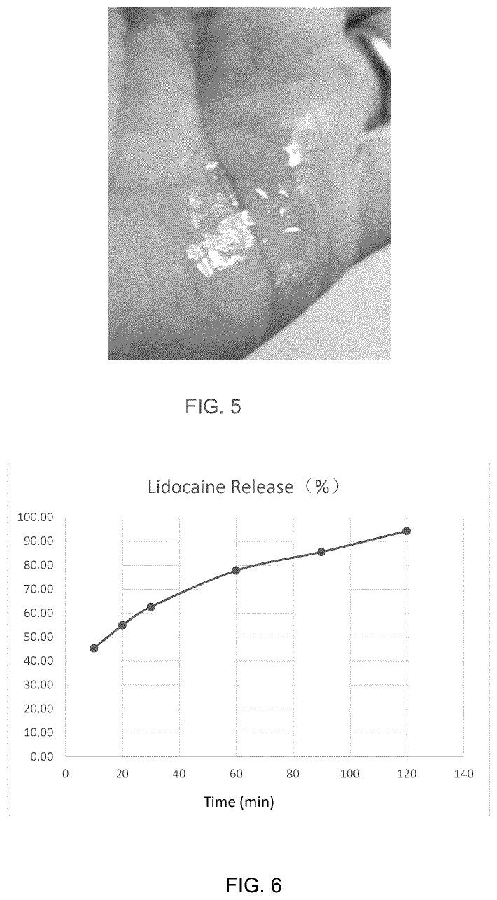

The sprayable in situ polymerizable collagen was prepared by mix in situ polymerizable collagen prepared in Example 1 with PBS at volume ratio of 2:1. After mixing by stirring at 50 rpm for 10 min, the gel was centrifuge to remove the bubbles. The sprayable in situ polymerizable collagen was less viscous with better flowability and placed in a spray bottle and still remained transparent under room temperature. And it was sprayed to skin, and formed a transparent film by contacting moist of skin as showed in FIG. 5.

### Example 7. Delivery of Liarozole for Intravaginal Administration

The in situ polymerizable collagen was prepared using an speed-up method using ultrafiltration instead of dialysis described by DeVore, et. al. in U.S. Pat. No. 10,111,981. Pure soluble Type I atelo-collagen lyophilized particles/powder was purchased from Nitta Gelatin and dissolved in 0.01M HCl solution at a concentration from 4.0-5.0 mg/mL.

An ultrafiltration was used to remove addition salt or small molecule salt. A MWCO of 10,000-14,000 Da ultrafiltration membrane was used in ultrafiltration. The ultrafiltration membrane can be ceramic membrane or polymer membrane package (including flat sheet membrane package, hollow fiber membrane module or spiral-wound membrane cartilage). 2.5 to 5 times volume of 0.01 M HCl solution was used to buffer the collagen solution and the volume of collagen solution remained the same before and after ultrafiltration. Collagen solution was sterile filtered to remove microbials and 0.15M EDTA-2Na sterile solution was added to the soluble collagen solution (4.5 mg/mL). Additional sterile PBS (20-24 g NaOH, 8.4 g Na₂HPO₄, 1.2 g NaH₂PO₄ in 1,000 mL water) or 0.15M Na₂CO₃ solution was added to the collagen solution to adjust pH to 6.5-7.6. The mix ratio of collagen solution, EDTA-2Na solution and pH adjust solution was 4:1:1.

The final clear in situ polymerizable collagen solution was collected and defoaming through negative pressure. Liarozole nano crystals was mixed to in situ polymerizable collagen solution to reach the concentration at 0.1 mg/mL to 1.0 mg/mL. The in situ polymerizable collagen solution with Liarozole was directly delivered by spraying into uterus through vagina using a special extension nozzle to treat uterine fibroids or endometriosis to avoid gastrointestinal side effects. The sprayable in situ polymerizable collagen is capable of releasing Liarozole up to 2 weeks with excellent resistance to vaginal secretion.

### Example 8. Delivery Epidermal Growth Factor by Sprayable In Situ Polymerizable Collagen

The in situ polymerizable collagen was prepared using a speed-up method using ultrafiltration instead of dialysis described by DeVore, et. al. in U.S. Pat. No. 10,111,981. Pure soluble Type I atelo-collagen lyophilized particles/powder was purchased from Nitta Gelatin and dissolved in 0.01M HCl solution at a concentration from 4.0-5.0 mg/mL. An ultrafiltration was used to remove addition salt or small molecule salt. A MWCO of 10,000-14,000 Da ultrafiltration membrane was used in ultrafiltration. The ultrafiltration membrane can be ceramic membrane or polymer membrane package (including flat sheet membrane package, hollow fiber membrane module or spiral-wound membrane cartilage). 2.5 to 5 times volume of 0.01 M HCl solution was used to buffer the collagen solution and the volume of collagen solution remained the same before and after ultrafiltration. Collagen solution was sterile filtered to remove microbials. Additional sterile EDTA-2Na and mannitol solution was added to the soluble collagen solution (4.5 mg/mL). The collagen solution was lyophilized to collect collagen solid foams.

The collagen foam was dissolved in water, and sterile PBS (24 g NaOH, 8.4 g Na₂HPO₄, 1.2 g NaH₂PO₄ in 1,000 mL water) was added to the collagen solution to adjust pH to 6.5-7.6. The final collagen concentration is 5 mg/mL with 0.25 mM EDTA-2Na and 3% mannitol.

The final clear in situ polymerizable collagen viscous solution was collected and defoaming through negative pressure. Epidermal Growth Factor lyophilized powder was dissolved in in situ polymerizable collagen solution to reach the concentration at 5,000 IU/mL˜20,000 IU/mL.

The in situ polymerizable collagen solution with EGF was sprayed to skin wounds through spray bottle and formed a film to cover the wound within 120 sec, providing a wet healing environment and continuous EGF delivery to accelerate skin healing or improve skin condition.

### Example 9. Short-Term Controlled Release of Lidocaine

The in situ polymerizable collagen was prepared using a speed-up method using ultrafiltration instead of dialysis described by DeVore, et. al. in U.S. Pat. No. 10,111,981. Pure soluble Type I atelo-collagen lyophilized particles/powder was purchased from Nitta Gelatin and dissolved in 0.01M HCl solution at a concentration from 4.0-5.0 mg/mL. An ultrafiltration was used to remove addition salt or small molecule salt. A MWCO of 10,000-14,000 Da ultrafiltration membrane was used in ultrafiltration. The ultrafiltration membrane can be ceramic membrane or polymer membrane package (including flat sheet membrane package, hollow fiber membrane module or spiral-wound membrane cartilage). 2.5 to 5 times volume of 0.01 M HCl solution was used to buffer the collagen solution and the volume of collagen solution remained the same before and after ultrafiltration. Collagen solution was sterile filtered to remove microbials. One third of the same volume of collagen solution of a lidocaine sterile solution (0.9% Lidocaine, 7% Mannitol, 80 mM EDTA-2Na, 0.05M Na₂CO₃ and 0.05M NaHCO₃) was added to collagen. 2 mL final mixed collagen solution was incubated under 37° C. to form a gel and put into 20 mL 37° C. 0.9% NaCl solution with a stir bar stirring at 80 RPM.

At 10 min, 20 min, 30 min, 60 min, 90 min, and 120 min, 2 mL NaCl solution was collected to test lidocaine concentration and additional 2 mL 0.9% NaCl solution was replenished. HPLC method was used to measured lidocaine concentration and calculated to the percentage release of the lidocaine in collagen gel. The data was presented in FIG. 6.

### Example 10. Coating Medical Device with In Situ Polymerizable Collagen/Antibiotic Composition

In situ polymerizable collagen/Voriconazole was prepared as described in Example 3. A dip-coating process was applied to the implantable screws. Spray-coating was applied to the screw and hernia-mesh parts to test for drug delivery coating. After drying, the screw (FIG. 7A) and hernia mesh parts (FIG. 7B) were well coated with collagen films.

The antibiotic property of coated screw was performed by cultured in 10 ml of trypticase soy broth as a medium in tubes with 100 CFU S. aureus (FIG. 7C #2). Other control included medium with bacteria (growth control, FIG. 7C #1), screw with in situ polymerizable collagen without Voriconazole in medium with bacteria (FIG. 7C #3), in situ polymerizable collagen without Voriconazole in medium with bacteria (FIG. 7C #4), and in situ polymerizable collagen alone without bacteria (FIG. 7C #5). The test tubes were cultured 35° C. for three days. The tube contained in situ polymerizable collagen/Voriconazole coated screw showed no bacteria growth and the other controls showed cloudiness (FIG. 7C). Voriconazole carried by in situ polymerizable collagen can be well-coated to medical device and showed antibacterial activity.

Although the present invention has been described with reference to exemplary embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention herein. Such equivalents are intended to be encompassed in the scope of the present invention.

All references, including patents, publications, and patent applications, mentioned in this specification are herein incorporated by reference in the same extent as if each independent publication, patent or patent application was specifically and individually indicated to be incorporated by reference.

1. A composition comprising:
(i) a neutral collagen solution, which is injectable or sprayable and capable of converting into a fibrous collagen mass or gel upon contacting with a physiological fluid or solution, or water; and
(ii) a biologically or pharmaceutically active agent in the neutral collagen solution.

2. The composition of claim 1, wherein the composition is an injectable and/or sprayable, in situ polymerizable collagen composition; and/or
wherein the source of collagen in the neutral collagen solution is selected from allogenetic, mammal hides or marine species or axolotl hides derived matrix; and/or
wherein the collagen in the neutral collagen solution is selected from full collagen or atelocollagen, or recombinant collagen peptides from microorganism, plants, insect cells or animal cells, or collagen mimic peptides; and/or
wherein the collagen is soluble at neutral pH, does not undergo fibrillogenesis in the solution, and polymerizes upon exposure to a tissue site in vivo or to a physiological fluid or solution or water in vitro or ex vivo; and/or
wherein the pH of the neutral collagen solution is from about 6.5 to about 7.5; and/or
wherein the pH of the physiological fluid or solution or water is from about 6.5 to about 7.5; and/or
wherein the neutral collagen solution comprises an acid soluble collagen, EDTA or EGTA or salts thereof, and optionally a polyol.

3. The composition of claim 2, wherein the collagen in the neutral collagen solution is in a concentration between 5 and 70 mg/ml; and/or
wherein the salt of EDTA is disodium EDTA or phosphate and/or citrate and/or carbonate and/or chloride salt of EDTA; and/or
wherein the salt of EGTA is disodium EGTA or phosphate and/or citrate and/or carbonate and/or chloride of EGTA; and/or
wherein EDTA or EGTA or salt thereof is in a concentration between 10 and 50 mM; and/or
wherein the polyol is a sugar alcohol; and/or
wherein the polyol is in a concentration between 2.5% and 4% (w/v).

4. The composition of claim 3, wherein the sugar alcohol is selected from D-mannitol; and/or
wherein the solution further comprises a disaccharide, fructose, or combinations thereof.

5. The composition of claim 1, wherein the active agent is selected from a drug, a biological molecule, a therapeutic agent, a diagnostic agent; and/or
wherein the active agent is selected from a chemical drug or active ingredient, a biological drug or active ingredient, a radioactive substance, a chemiluminescent or bioluminescent active substance; and/or
wherein the active agent is in the form of an element, a compound, a composition, a polymer, an oligomer, a conjugate or combinations thereof; and/or
wherein the active agent is in a further controlled release form selected from the group consisting of polymer modification, polymer encapsulation, nano-crystallization, microsphere formation, lipid encapsulation, lipid modification, lyophilization, emulsification; and/or
wherein the active agent is selected from the group consisting of a digestive tract drug, metabolic drug, blood and hematopoietic organ drug, cardiovascular system drug, skin drug, genitourinary system drug, hormone drug, systemic anti-infection drug, anti-tumor drug, immunomodulator drug, musculoskeletal system drug, nervous system drug, anti-parasitic drug, respiratory system drug, sensory organ drug; and/or
wherein the active agent is a drug for treating disease selected from a peptide or protein, a nucleotide drug, a steroid, an antibiotic, an angiogenic agent, an inhibitor of angiogenesis, or an inhibitor of cell proliferation, an immune inhibitor, an antibiotics, an enzyme inhibitor, a signaling receptor agonist, a signaling receptor antagonist, a signaling receptor blocker, a chemotherapy drug, a radioactive drug, a hormone drug, a toxin drug, or a cell-based product; and/or
wherein the active agent is selected from latanoprost, peptide, steroids, cyclosporin voriconazole, ¹²⁵-I, liarozole and EGF, atropine or botulinum toxin.

6. The composition of claim 1, wherein the concentration of the active agent in the neutral collagen solution is ranged from 1 μg/ml to 100 mg/ml; and/or
wherein the active agent is solved or dispersed in the collagen solution, entrapped in the collagen, or covalently bound and crosslinked to the collagen; and/or
wherein the active agent is homogeneously mixed with the neutral soluble collagen solution.

7. The composition of claim 1, wherein the composition is in a form suitable for administering to a target or a part thereof by injecting, spraying, topical application, coating or dispersing; and/or
wherein upon administered to a target, the solution converted to a fibrous mass or gel carrying, delivering and/or sustained releasing the active agent; and/or
wherein upon administration, the composition converts to a fibrous mass or gel covering the tissue, blocking the vessels or canals or packing the wound; and/or
wherein upon administration, the composition converts to a fibrous mass or gel within 180 second; and/or
wherein the active agent is sustained released in at least 1 day, at least 2 days, at least 1 week, at least 2 weeks, at least a month, at least 2 months, at least 3 months, at least 6 months; and/or
wherein the tissue is selected from one or more of ocular, otic, nasal, bone, cartilage, meniscus, tendon, ligament, dermis or vagina.

8. The composition of claim 7, wherein the target is a living human or animal or a part thereof, a medical device, a cosmetic device, and/or wherein the physiological fluid or solution or water is in a tissue site in vivo or is added to the collagen solution in vitro; and/or
wherein the tissue is selected from the group consisting of sclera, vitreal, conjunctival, sub tenon's, retinal, cochlea, the endolymphatic sac or duct, the vestibular labyrinth, and all of the compartments and connecting tubes which include these components, sinus tissues; and/or
wherein the tissue site is epidermis, dermis acute or chronic wounds, mucosa or submucosa wounds or disorder, and soft tissue wounds during surgery; and/or
wherein the tissue is or comprises orifices, respiratory tract, digestive tract or genital tract; and/or
wherein the physiological fluid or solution is a body liquid or an artificial physiologically acceptable liquid; and/or
wherein the physiological fluid or solution is selected from blood, plasma, interstitial fluid, lymph fluid, cerebrospinal fluid, saline, Ringer's solution and derivates thereof, hydroxyethyl starch (HES), dextran, Tyrode's solution, PBS, Hank's Balanced Salt Solution (HBSS), Earle's Balanced Salt Solution (EBSS).

9. A method for sustained delivery of a biologically or pharmaceutically active agent to a target in need thereof, wherein the method comprises: (a) providing a composition comprising the active agent in a neutral collagen solution, wherein the neutral collagen solution is injectable or sprayable and capable of converting into a fibrous collagen mass or gel upon contacting with a physiological fluid or solution, or water; and (b) administering the composition to the target, whereby, upon contact with a physiological fluid or solution or water, the solution converts to a gel or fibrous mass and the active agent is sustained released to the target.

10. The method of claim 9, wherein the target is a living human or animal or a part thereof, a medical device, a cosmetic device or a part thereof; and/or
wherein the target is a tissue site for treatment of a disease or wound via or in a tissue site; and/or
wherein the composition is an injectable and/or sprayable, in situ polymerizable collagen composition; and/or
wherein the source of collagen in the neutral collagen solution is selected from allogenetic, mammal hides or marine species or axolotl hides derived matrix; and/or
wherein the collagen in the neutral collagen solution is selected from full collagen or atelocollagen, or recombinant collagen peptides from microorganism, plants, insect cells or animal cells, or collagen mimic peptides; and/or
wherein the collagen is soluble at neutral pH, does not undergo fibrillogenesis in the solution, and polymerizes upon exposure to a tissue site in vivo; and/or
wherein the pH of the neutral collagen solution is from about 6.5 to about 7.5; and/or
wherein the neutral collagen solution comprises an acid soluble collagen, EDTA or EGTA or salts thereof, and optionally a polyol.

11. The method of claim 10, wherein the collagen in the neutral collagen solution is in a concentration between 5 and 70 mg/ml; and/or
wherein the salt of EDTA is disodium EDTA or phosphate and/or citrate and/or carbonate and/or chloride salts of EDTA; and/or
wherein the salt of EGTA is disodium EGTA or phosphate and/or citrate and/or carbonate and/or chloride salts of EGTA; and/or
wherein EDTA or EGTA or salt thereof is in a concentration between 10 and 50 mM; and/or
wherein the polyol is a sugar alcohol; and/or
wherein the polyol is in a concentration between 2.5% and 4% (w/v).

12. The method of claim 11, wherein the sugar alcohol is selected from D-mannitol;
and/or wherein the solution further comprises a disaccharide, fructose, or combinations thereof.

13. The method of claim 9, wherein the active agent is selected from a drug, a biological molecule, a therapeutic agent, a diagnostic agent; and/or
wherein the active agent is selected from a chemical drug or active ingredient, a biological drug or active ingredient, a radioactive substance, a chemiluminescent or bioluminescent active substance; and/or
wherein the active agent is in the form of an element, a compound, a composition, a polymer, an oligomer, a conjugate or combinations thereof; and/or
wherein the active agent is in a further controlled release form selected from the group consisting of polymer modification, polymer encapsulation, nano-crystallization, microsphere formation, lipid encapsulation, lipid modification, lyophilization, emulsification; and/or
wherein the active agent is selected from the group consisting of a digestive tract drug, metabolic drug, blood and hematopoietic organ drug, cardiovascular system drug, skin drug, genitourinary system drug, hormone drug, systemic anti-infection drug, anti-tumor drug, immunomodulator drug, musculoskeletal system drug, nervous system drug, anti-parasitic drug, respiratory system drug, sensory organ drug; and/or
wherein the active agent is a drug for treating disease selected from a peptide or protein, a nucleotide drug, a steroid, an antibiotic, an angiogenic agent, an inhibitor of angiogenesis, or an inhibitor of cell proliferation, an immune inhibitor, an antibiotics, an enzyme inhibitor, a signaling receptor agonist, a signaling receptor antagonist, a signaling receptor blocker, a chemotherapy drug, a radioactive drug, a hormone drug, a toxin drug, or a cell-based product or vaccine; and/or
wherein the active agent is selected from latanoprost, peptide, steroids, cyclosporin voriconazole, ¹²⁵-I, liarozole and EGF, atropine or botulinum toxin.

14. The method of claim 9, wherein the concentration of the active agent in the neutral collagen solution is ranged from 1 μg/ml to 100 mg/ml; and/or
wherein the active agent is solved or dispersed in the neutral soluble collagen solution, entrapped in the collagen, or covalently bound and crosslinked to the collagen; and/or
wherein the active agent is homogeneously mixed with the neutral soluble collagen solution; and/or
wherein the composition is in a form suitable for administering to a target by injecting, spraying, topical application, coating or dispersing; and/or
wherein upon administered to a tissue site of a target, the solution converted to a fibrous mass or gel carrying, delivering and/or sustained releasing the active agent; and/or
wherein upon administration, the composition converts to a fibrous mass or gel covering the tissue, blocking the vessels or canals or packing the wound; and/or
wherein upon administration, the composition converts to a fibrous mass or gel within 180 second; and/or
wherein the active agent is sustained released in at least 1 day, at least 2 days, at least 1 week, at least 2 weeks, at least a month, at least 2 months, at least 3 months, at least 6 months; and/or
wherein the tissue is selected from one or more of ocular, otic, nasal, bone, cartilage, meniscus, tendon, ligament, dermis or vagina.

15. The method of claim 9, wherein the physiological fluid or solution or water is in a tissue site in vivo or is added to the collagen solution in vitro; and/or
wherein the tissue is selected from the group consisting of sclera, vitreal, conjunctival, sub tenon's, retinal, cochlea, the endolymphatic sac or duct, the vestibular labyrinth, and all of the compartments and connecting tubes which include these components, sinus tissues; and/or
wherein the tissue site is epidermis, dermis acute or chronic wounds, mucosa or submucosa wounds or disorder, and soft tissue wounds during surgery; and/or
wherein the tissue is or comprises orifices, respiratory tract, digestive tract or genital tract; and/or
wherein the physiological fluid or solution is a body liquid or an artificial physiologically acceptable liquid; and/or
wherein the physiological fluid or solution is selected from blood, plasma, interstitial fluid, lymph fluid, cerebrospinal fluid, saline, Ringer's solution and derivates thereof, hydroxyethyl starch (HES), dextran, Tyrode's solution, PBS, Hank's Balanced Salt Solution (HBSS), Earle's Balanced Salt Solution (EBS
